(19) United States
(12) Patent Application Publication
(10) Pub. No.: US 2024/0145044 A1
(43) Pub. Date: May 2, 2024

(54) CONTEXTUAL AWARENESS FOR UNSUPERVISED ADMINISTRATION OF COGNITIVE ASSESSMENTS REMOTELY OR IN A CLINICAL SETTING

(71) Applicant: Linus Health, Inc., Boston, MA (US)

(72) Inventors: Shamay Agaron, Boston, MA (US); Sean Tobyne, Boston, MA (US); Shashank Manjunath, Boston, MA (US)

(21) Appl. No.: 18/374,250

(22) Filed: Sep. 28, 2023

Related U.S. Application Data
(60) Provisional application No. 63/377,435, filed on Sep. 28, 2022.

(51) Int. Cl. G16H 10/20; A61B 5/11; G06N 3/08; G06N 5/04

(52) U.S. Cl. CPC G16H 10/20; A61B 5/1125; G06N 3/08; G06N 5/04; A61B 2560/0242

(57) ABSTRACT

According to various embodiments, a solution including methods, systems, and computer program products is provided for assessing environmental context around an individual taking an assessment. In various embodiments, a method of assessing an individual is provided. A plurality of signals, each signal from one sensor of a plurality of sensors, are received. Each signal may be associated with a modality of assessment. Each of the plurality of signals may be processed with an individualized signal processing module. A plurality of features, each from one of the processed plurality of signals, may be extracted. The plurality of features may be aggregated into a machine learning input with a feature processing module. The machine learning input may be provided to a machine learning algorithm. That environmental interference is occurring may be inferred based on the output of the machine learning algorithm.

### CROSS-REFERENCE TO RELATED APPLICATION(S)

This application claims the benefit of priority of U.S. Provisional Application No. 63/377,435, filed Sep. 28, 2022, which is hereby incorporated by reference in its entirety.

### BACKGROUND

Remote administration of health and cognitive assessments has received a great deal of attention due to recent advancement in technology, the COVID-19 pandemic, and resulting increase in telehealth use in clinical practice and clinical trials. The benefits conveyed by remote assessments include: (1) the possibility for more frequent data gathering, (2) no (or reduced) travel requirements for patients and/or study participants, (3) greater access to patients and/or study participants, and (4) the mitigation of health disparities among underserved communities. Thus, remote unsupervised administration of cognitive assessments enables greater access to healthcare for patients and greater access to study populations for clinical studies (e.g., pharmaceutical trials), but faces the challenge that the patient and/or participant is not in a controlled environment under the observation of a trained professional. Furthermore, in today's busy healthcare clinic practices, there is a shortage of providers with adequate time to observe the entire assessment. By leveraging the sensors in the digital device to record distractions and cues that would otherwise be noted by healthcare professionals, the need for professionals' presence during the entire duration of the assessment can be obviated. There is, therefore, a need for cognitive assessments that consider how the environment of the participant may affect assessment results and, based on observations, identify objectives to take corrective actions to improve assessment quality and/or accuracy.

### BRIEF SUMMARY

According to some embodiments of the present disclosure, systems, methods, and computer program products are provided for assessing an individual, such as a patient/participant. In various embodiments, a method for assessing environmental context around an individual taking an assessment is provided. A plurality of signals, each signal from one sensor or a plurality of sensors, are received. Each signal is associated with at least one modality of assessment. Each of the plurality of signals is processed with an individualized signal processing module. A plurality of features, each from one of the processed plurality of signals, are extracted. The plurality of features is aggregated into a neural network input or other signal processing, and/or machine-learning algorithm input with a feature-processing module. The input is provided to a neural network, machine-learning model, or other algorithm. The environmental interference that is occurring is inferred based on the output of the neural network or other algorithm.

In various embodiments, a method for assessing environmental context around an individual taking an assessment is provided. A plurality of signals, each signal from one sensor of a plurality of sensors, are received. Each signal is associated with at least one modality of assessment. Each of the plurality of signals is processed with an individualized signal-processing module. A plurality of features, each from one of the processed plurality of signals, are extracted. The plurality of features are aggregated into a machine learning input with a feature processing module. The machine-learning input is provided to a machine learning algorithm. That environmental interference is occurring is inferred based on the output of the machine learning algorithm.

In various embodiments, a system is provided including a computing node comprising a computer readable storage medium having program instructions embodied therewith. The program instructions are executable by a processor of the computing node to cause the processor to perform a method. A plurality of signals, each signal from one sensor of a plurality of sensors, are received. Each signal is associated with at least one modality of assessment. Each of the plurality of signals is processed with an individualized signal processing module. A plurality of features, each from one of the processed plurality of signals, are extracted. The plurality of features is aggregated into a machine learning input with a feature processing module. The machine learning input is provided to a machine learning algorithm. That environmental interference is occurring is inferred based on the output of the machine learning algorithm.

In various embodiments, a computer program product for assessing environmental context in which an individual takes an assessment is provided including a computer readable storage medium having program instructions embodied therewith. The program instructions are executable by a processor to cause the processor to perform a method. A plurality of signals, each signal from one sensor of a plurality of sensors, are received. Each signal is associated with at least one modality of assessment. Each of the plurality of signals is processed with an individualized signal processing module. A plurality of features, each from one of the processed plurality of signals, are extracted. The plurality of features are aggregated into a machine learning input with a feature processing module. The machine learning input is provided to a machine learning algorithm. That environmental interference is occurring is inferred based on the output of the machine learning algorithm.

### BRIEF DESCRIPTION OF THE DRAWINGS

FIG. 1 depicts a block flow diagram of a system for assessing individuals according to various embodiments of the present disclosure.

FIG. 2 is a flow diagram of example technique for assessing an individual according to various embodiments of the present disclosure.

FIG. 3 depicts a computing node according to various embodiments of the present disclosure.

### DETAILED DESCRIPTION

The participant's environment can influence diagnostic assessment results in three key ways. First, the environment in which the assessment is taken may introduce artifacts into mobile device sensors during an assessment, such as by the device being misused or the assessment not being performed according to instructions. Second, the environment may distract a participant or impact their cognitive and/or emotional state during an assessment and affect how they perform. Third, assessment of the individual while taking the test (e.g. are they squinting to see, turning their head to hear, looking around the room, et. al.) can lead to greater phenotypic or diagnostic understanding (i.e., vision, hearing, or attention deficit issues, etc.), which provides the clinician with a clear understanding of the environment in which the assessments are taken. These first two effects may be known as ‘environmental interference’ and the latter one could be ‘performance deficits’.

An assessment may depend on specific modalities, such as voice and/or speech assessments recording audio data, visuoconstructional assessments recording drawing data, motion, gait and/or balance assessments recording device movement data, eye movements, facial-expression analysis, and the like. Similarly, sources of environmental interference may also have dominant modalities. The modality of the diagnostic assessment may determine how it may be affected by the modality of environmental interference. For instance, a diagnostic assessment based on tracking a participant's gaze location on a mobile device, i.e., eye tracking, may involve video analysis algorithms that are adversely affected by insufficient light. Those same algorithms may suffer no problematic effects from audio interference in the environment, such as elevated background noise, microphone hardware malfunction, or the like. However, an assessment based on recording and analyzing a participant's voice and speech may be degraded by audio interference.

Interference may not always be recorded directly by sensor input on mobile devices during assessments. A participant may be distracted by interference in their environment, which affects their performance. For example, during an eye-tracking assessment, a participant may look off-screen at a family member entering the room. While the family member may not appear in the video input, the participant's performance would be affected by lapses of attention and the eye tracking algorithm results would be skewed as a result. Furthermore, at times and with the best intentions, family members may try to “help” individuals taking the test such as reminding them of the words in a memory test, which can invalidate the results of a memory test. Detecting and analyzing this type of environmental distraction (changes in patterns of an activity, or changes in the source of input in the example of a difference in voice being perceived in a recording) is also important for managing data quality.

Environment interference may cause particular assessments to fail entirely or they may skew the results. Being able to detect the latter is imperative to prevent misdiagnosis. For instance, a participant may be misdiagnosed as having cognitive impairment because the traffic outside their window was distracting them as they were trying to complete a time-sensitive eye-tracking portion of the assessment.

Corrective actions may be taken to mitigate the environmental effects of the assessment results. Corrective actions can be considered to fall into two high-level example categories: A first high-level category may be one in which the assessment(s) cannot or should not proceed based on contextual awareness. For such assessment(s), recommendations may be provided to modify the environmental situation. For example, if the system determines that the background noise is too high, it may prompt the user to move to a quieter setting before proceeding. A second high-level category may be a situation where assessment(s) can proceed but will likely be influenced by the environment. For such assessment(s), contextual awareness can be leveraged to correct for environmental influences before data analysis begins. For example, the system may adjust the scoring, or if there is a valid way to do so, adjust the data processing to remove noisy components, or the system may mark an assessment result as low quality, prompting the interpreter of the results to review the assessment carefully and manually score the assessment.

A few conventional solutions have focused on deep learning algorithms for Human Activity Recognition (HAR). However, these solutions and algorithms have several limitations for remote participant monitoring applications. In particular, these conventional solutions and algorithms are primarily focused on single context recognition, and cannot handle multiple distractions at the same time. For example, such solutions and algorithms would not be able to identify that a participant was both walking and talking at the same time while performing a remote assessment. Instead, such solutions and algorithms would only be able to identify a single context, walking or talking. In addition, the conventional solutions and algorithms are evaluated on datasets which leverage scripted scenarios on a limited set of contexts, such as six contexts, with a limited number of similar participants, such as nine participants in the 25-30 age range. While these results provide a useful baseline for algorithm performance, it remains to be seen whether the algorithms extend to larger datasets collected during daily device use, i.e., “in-the-wild,” rather than in scripted scenarios. Additionally, many datasets, on which the conventional solutions and algorithms are evaluated, leverage inertial measurement unit (IMU) data collected using contact sensors at multiple points on the body. Determination of contextual awareness during remote health assessment, however, is a more difficult task because all IMU measurements will likely be taken on a single device that is not necessarily attached to the body.

Yet other conventional solutions have focused on mobile phones and single modalities of environmental interference. Additionally, some conventional solutions have focused on determination of multiple co-occurring physical activities from smartphone data measurements. In particular, these solutions have attempted to elucidate individual potentially co-occurring physical activities. The aforementioned conventional solutions are insufficient to fully assess contextual awareness during remote assessments given the complexity of the multi-source signals. The need for multiple modalities for problems such as gait detection or fine-grain physiologic monitoring has been established, such as by work related to HAR.

The diagnostic assessment solution, as presented herein, considers multiple modalities, increases accuracy of diagnostics, and allows for improved inference of device context. The diagnostic solution may be capable of correlating potential distractions across multiple sensing modalities using a single, overarching algorithm which can simultaneously process multiple incoming sensor signals. The solution as presented herein operates to measure contextual awareness from low-level sensor data and detecting human physical activities. The solution as presented herein may operate on multiple modalities for a variety of problems, such as gait detection, fine-grain physiologic monitoring, and/or the like.

The solution as presented herein includes systems, a framework, and related algorithms that can perform a variety of operations. In particular, the solution can detect the environmental context of an individual participant including potential distractions and artifacts introduced to sensor inputs that may influence the participant's performance or skew assessment results when the participant is interacting with a remote application that may involve assessments of multiple input modalities, such as audio and video. The solution may flexibly incorporate multiple streams of information and make inferences about multiple sources of environmental interference. The solution may also determine which assessments are affected and in what manner by the environmental situation based on both the modality of the assessment as well as the modality of the environmental distractions. The solution may additionally recommend or take corrective action based on a prior determination.

The solution as presented herein has several advantages. For example, the framework associated with the solution can improve accuracy of multimodal algorithms by ameliorating environmental interferences based on the effects the interferences have on different modalities. As a consequence, the framework improves the accuracy of assessment results in remote settings. As another example, the framework associated with the solution includes innovations in recommendations based on contextual awareness as well as novel algorithmic techniques that combine both deep learning and traditional signal processing techniques for correcting noise from environmental effects. As a consequence, the framework again improves accuracy with remote assessments.

### Detecting Environmental Interference and Classifying it for Modal Contextual Awareness

Environmental conditions that affect contextual awareness may be unimodal or multimodal. Unimodal conditions may be conditions such as high background noise or background speech, which may be detected by a single sensor, such as a microphone. Multimodal conditions are conditions such as walking or driving in conjunction with speaking, which require multiple sensors, such as an accelerometer, a gyroscope, a microphone, a video camera, or the like to detect.

Mitigation of interference in remote assessment administration may occur in two steps. The first step may be to detect that interference is occurring. The second step may be to classify the type of interference and apply corrective measures. The corrective measures may include flagging the data for additional processing or repetition of the assessment with mitigation of the interference source.

The solution, as described herein, performs both steps and uses novel algorithms to detect interference. Once interference is detected by the solution, data is flagged by the solution. Depending on the type of interference, the participant may be asked to repeat the task or for confirmation that the detected form of interference occurred.

Mobile device sensors may be able to directly observe the environment and to measure interference for each input modality. Examples of direct measure of potential interference may include: a microphone measuring ambient noise within the room where a participant is located, a camera measuring ambient light within the room where a participant is located, an accelerometer measuring acceleration to determine whether the device is stationary or moving, and/or the like.

Described herein are methods, systems, and computer program products associated with a solution for assessing an individual, such as a participant. In various embodiments, the solution may be a stand-alone tool that may not rely on existing solutions. The solution, as described herein, may be referred to as an agent. In various embodiments, the solution may be implemented using hardware and/or software, and may be able to interface with a participant for the purposes of assessing the participant.

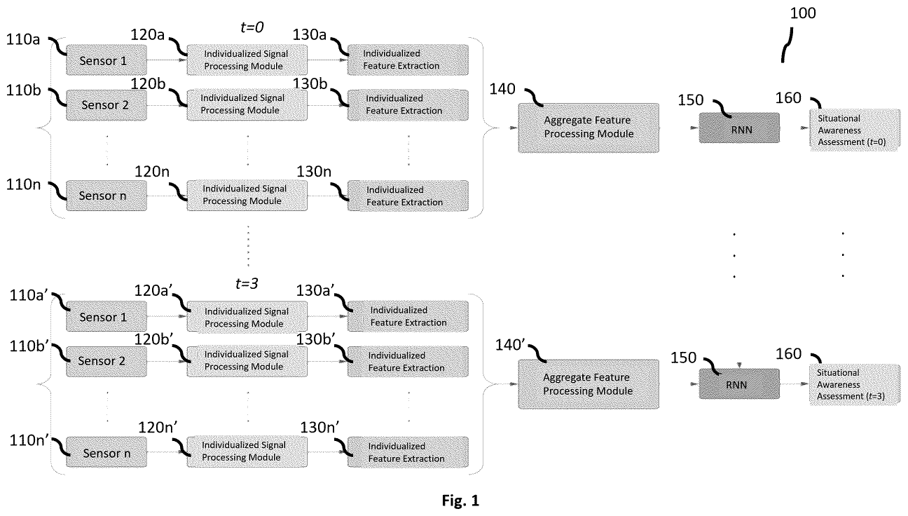

FIG. 1 depicts a block flow diagram 100 of a system for assessing individuals, in accordance with various embodiments of the present disclosure. The solution, as described herein, may use an algorithm that includes several steps. First, multiple signals may be received. Each of the signal(s) may be from one of multiple sensors, such as sensors 110a, 110b, . . . , or 110n. Each of the signal(s) may be associated with a modality of assessment. For example, accelerometer signals may be received from an accelerometer, gyroscope signals may be received from a gyroscope, audio signals may be received from a microphone, and/or video signals may be received from a video camera.

Each of the multiple signals may be processed by a signal processing module, such as an individualized signal processing module 120a, 120b, . . . , or 120n. In particular, low-level signal processing may be performed on each of the incoming/received signals. Such low-level signal processing may extract feature(s) pertaining to environmental interference. Each individualized signal processing module may be tuned to process signal(s) received from a specific sensor type.

For example, accelerometer signals may be transformed, by an individualized signal processing module tuned for accelerometer signals. The accelerometer signals may be transformed into the spectral domain using a Discrete Fourier Transform (DFT) before being input into an interference detection algorithm. The DFT may be performed over a predetermined window size, such as 1-10 seconds. As another example, gyroscope signals may be transformed, by an individualized signal processing module tuned for gyroscope signals. The transformation of the gyroscope signals may be performed in a similar manner as the aforementioned transformation of accelerometer signals. As yet another example, audio signals may be transformed, by an individualized signal processing module tuned for audio signals. The audio signals may be transformed into a spectrogram. This may allow for the association of frequency with time at a finer scale than a standard DFT allows. As yet another example, video signals may be grouped together, by an individualized signal processing module tuned for video signals, over a predetermined window size, such as 1-10 seconds, and passed directly to the feature extraction module. Such a per-sensor processing framework may allow for easy addition of further signals as sensor technology develops, such as when mobile sensor technology develops.

Once each of the signals is processed using the individualized signal processing module, each result may be input to an extraction module, such as individualized feature extraction module 130a, 130b, . . . , or 130n. In particular, once each individual sensor type has been processed into a low level feature by the individualized signal processing modules 120, the interference detection algorithm may process each low-level feature into a higher level feature. This may be performed using stackable feature extraction modules 130. Specifically, each individual low-level feature may be processed by an individualized feature extraction module 130a, 130b, . . . , or 130n. Each of the individualized feature extraction modules 130 may use a deep neural network architecture to perform its processing. Each of the individualized feature extraction module 130 may extract multiple higher level features, each from one of the signals processed by individualized signal processing module 120. The output of each feature extraction modules 130 may be a single high-level feature vector associated with one of the input sensors 110.

For example, the aforementioned accelerometer signals output by one of the individualized signal processing modules 120 may be processed by a 1D convolutional neural network within one of the individualized feature extraction modules 130. The 1D convolutional neural network may be operating on spectral domain data, and may output latent vector(s), such as n n-dimensional latent vectors, where n is a natural number. As another example, the aforementioned gyroscope signals output by one of the individualized signal processing modules 120 may be processed by one of the individualized feature extraction modules 130 in a similar manner as described above for accelerometer signals. As yet another example, audio data, which may be processed into a spectrogram by one of the individualized signal processing modules 120, may then be processed by a 2D convolutional neural network within one of the individualized feature extraction modules 130. The 2D convolutional neural network may output latent vector(s), such as a 128-dimensional latent vector. As yet another example, video signals output by one of the individualized signal processing modules 120 may be processed frame-by-frame by a 2D convolutional neural network within one of the individualized feature extraction modules 130. The 2D convolutional neural network may output latent vector(s), such as a 16-dimensional latent vector for each frame. These latent vectors may then be concatenated together to form a 16m-dimensional vector, where m is the number of frames leveraged for the selected time window, and where m may be variably determined according to acquired frame rate.

The output of each feature extraction module 130 may be a single high-level feature vector, such as the latent feature vector(s) described above. The high-level feature vectors from each of the feature extraction modules 130 may be input to an aggregate feature processing module 140. Aggregate feature processing module 140 may operate to aggregate the feature vectors from the modules 130, and may output a single multi-dimensional feature vector, such as a single 128-dim feature vector. In various embodiments, aggregate feature processing module 140 may be a self-attention module. The aggregate feature processing module 140 may process data for a particular time instance, such as a first time instance, t=0.

Similar to what is described above, as shown in FIG. 1, sensors 110′, which may include 110a′, 110b′, . . . , and 110n′, where n is a natural number, may be similar in form and function to sensors 110, but may operate at a different time instance, such as t=3. Also as shown in FIG. 1, individualized signal processing modules 120′, which may include 120a′, 120b′, . . . , and 120n′, where n is a natural number, may be similar in form and function to individualized signal processing modules 120, but may operate at a different time instance, such as t=3. Further as shown in FIG. 1, individualized feature extraction modules 130′, which may include 130a′, 130b′, . . . , and 130n′, where n is a natural number, may be similar in form and function to individualized feature extraction modules 130, but may operate at a different time instance, such as t=3. Additionally, as shown in FIG. 1, aggregate feature processing module 140′ may be similar in form and function to aggregate feature processing module 140, may operate at a different time instance, such as t=3. In various embodiments, the plurality of features from aggregate feature processing module 140 and/or aggregate feature processing module 140′ may be aggregated into one or more signals, such as for a neural network input, such as recurrent neural network 150, for each time instance in a window of time instances.

The single multi-dimensional feature vector, such as a single 128-dim feature vector, output by aggregate feature processing module 140 may be input to a neural network, such as recurrent neural network 150. This may be performed at consecutive time instances in a period of time, such as t=1, t=2, t=3, etc. In particular, recurrent neural network 150 may process the feature vector input at time instance t=1, then it may process the input at time t=2, and then it may process the input at time t=3, etc. Recurrent neural network 150, may be updated at each time instance, and may output its result as situational awareness assessment data 160 at each time instance. In various embodiments, situational awareness assessment data 160 may be a single output feature vector. In various embodiments, situational awareness assessment data 160 may be processed using a self-attention model to produce a final output prediction.

The recurrent neural network 150 may allow for the association of small windows of signals, which may lead to more accurate predictions over time. For example, walking and jogging may have similar low-level signal characteristics over short time windows; however, over multiple time windows, the difference may be distinguishable. The feature extraction and classification technique, as presented herein, such as in FIG. 1, may be useful for identifying individual actions or environmental contexts.

The solution as presented herein, including the system and techniques presented in FIG. 1, is focused on the detection of participant distraction in remote assessment administration. In particular, the recurrent neural network 150 may be formulated to output potentially co-occurring specific distraction types, such as “walking” or “eyes away from screen,” or both at the same time. To this end, the recurrent neural network 150 may output a k-dimensional vector, where k is a natural number indicating the number of identified distractions.

Each individual element of the k-dimensional vector may have a value between 0 and 1, indicating how likely it is that the individual distraction, such as that caused by environmental interference, is occurring. This may allow for the thresholding of the individual distractions, and the detection of co-occurring distractions. For example, predetermined threshold(s) may be set and it may be determined that an individual distraction is occurring if the one or more elements of the k-dimensional vector have a value beyond the predetermined threshold(s). As another example, dynamic threshold(s), possibly based on environmental factors, may be set and it may be determined that an individual distraction is occurring if the one or more elements of the k-dimensional vector have a value beyond the predetermined threshold(s). The overall focus of the remote participant assessment solution is to produce an accurate assessment of a participant/individual. Therefore, in addition to detecting distractions, once the solution infers that environmental interference is occurring, the solution can apply and/or suggest an appropriate corrective action. Although various neural network input(s) and/or various neural network(s) are referenced herein, it should be understood that, in place of these, any machine learning input and machine learning model/algorithm, respectively, may be used without departing from the scope and spirit of what is described herein.

### Example Target Environmental Interference Distractions and Interventions

During individual assessments, there are many examples of possible environmental interference/distractions and of interventions that can be taken for such environmental interference/distractions. A first example may be when an individual participant is walking or otherwise moving while taking an assessment. In this case, the solution, as described herein, can leverage the aforementioned deep learning model(s) to identify motor activities during the assessment. The solution may then flag the data, and may ask the participant if they were walking, running, or participating in another motor activity during the assessment. If the participant responds affirmatively, then the participant may be asked to repeat the assessment and new data may be acquired by the solution. If the participant responds negatively, the data may be kept but flagged as having potential environmental interference using a boolean indicator or other assessment quality score, for example.

A second example may be when an individual participant is driving or a passenger in a car while taking an assessment. The solution may handle this case in a similar way as in the first example. In particular, the solution may ask the participant if they were in a car or driving, and ask the participant to repeat the assessment if the response is affirmative. If the response is negative, the solution may keep the data but may flag it for potential interference using a boolean indicator or assessment quality score, for example.

A third example may be when an individual participant holds the device in their hands or laps instead of securing it on a flat surface while taking an assessment. In this case, the solution can leverage the aforementioned deep learning model(s) to identify any movement during the assessment. The solution may then ask the participant to place the device on a flat surface and redo the assessment if possible. If this is not possible, the solution may keep the data but may flag it for potential interference using a boolean indicator or assessment quality score, for example.

A fourth example may be when there is excessive background noise when taking an assessment. In the case where there may be excessive background noise, there may be several different options. The first option may be to have the solution ask the participant to mitigate the background noise, such as through closing a window or moving to a quieter location. If this is not possible, as a second option, the solution may attempt to use a common mode rejection algorithm to subtract background noise detected in previous assessments from the background noise found in current assessments, in order to better isolate the signal. In various embodiments, such a common mode rejection algorithm may apply to an audio processing signal to improve the quality of the audio signal, although it may or may not eliminate noise heard by the participant. In addition, in various embodiments, such a common mode rejection algorithm may

Detecting Performance Deficits, Classifying them for Modal Contextual Awareness, and Providing Recommendations for Interventions

Contextual Awareness may capture information about the setting (context) in which a given individual performs a given task, thus enhancing the sensitivity of the task based insights and the value of the derived recommendations.

As a first example, an individual participant may be completing an assessment while traveling in a car, or a moving train. The accelerometers embedded in the testing device can detect the motion of a moving vehicle. Contextual awareness interpretation of the findings may appropriately consider the movement of the device to avoid false positive indications of tremor or motor control problems in the participant. Instead, a specific recommendation, made by the systems and/or methods herein, may include repeating a task while stationary and a comparison across conditions may enable more accurate evaluation.

As a second example, a loud environment may lead to a repeated word being hard to hear or an individual participant being asked to say a given work (e.g., ‘captain’) instead saying a different word (e.g., ‘carton’). Assessment of environmental noise can be informative as to whether hearing might be cause of the problem rather than an issue with an individual participant's memory. Assessment of environmental noise can also be informative as to whether distraction due to an environmental sound might have caused the mistake rather than an issue with an individual participant's hearing or memory problems. Specific recommendations, made by the systems and/or methods herein, may include sustained attention and distraction suppression tasks to further evaluate prefrontal and attentional networks.

As a third example, if an individual participant is turning up the volume or tilting their head and ear toward the device, the individual participant may be flagged as having hearing problems. Specific recommendations, made by the systems and/or methods herein, may include an audiology evaluation. Adaptive testing may offer a hearing screening test to further clarify the situation.

As a fourth example, detection that an individual participant is not wearing spectacles but is squinting, which may be indicated by measuring distance changes between eyelids, forehead muscle contraction, palpebral fissure width, and/or the like, could be flagged for possible vision problems associated with an individual participant. Such scenarios would need to be properly considered in the interpretation of cognitive task performance metrics, such as visuospatial perceptual or constructional abilities. Recommendations, made by the systems and/or methods herein, may include an optometry evaluation. Further testing for visual acuity or visual contrast sensitivity evaluations may be offered.

As a fifth example, if an individual participant's eye gaze keeps leaving the display, they could be distracted by an environmental factor (e.g., a noise which would be picked up by the microphone, a lighting change, and/or moving object/person which would be picked up by camera). Detecting such instances may provide a metric of attentional engagement and detection of distractions to better interpret and weigh results. It will also provide means to assess excessively easy distractibility and thus detect problems with sustained attention. Specific recommendations, made by the systems and/or methods herein, may include attention and distraction suppression tasks to further evaluate attentional network function.

As a sixth example, if an individual participant is walking while taking an assessment, systems and/or methods described herein may indicate that there is distraction of the participant However, such a scenario may also provide insight into the person's cognitive reserve, should there be a decline observed in cognitive performance while simultaneously walking. Similarly, detection, by the systems and/or methods described herein, of an individual participant talking or listening to music during task performance may allow analysis and interpretation of the results in the context of a dual task condition and this may allow for the extraction of metrics of cognitive reserve. Specific recommendations, made by the systems and/or methods herein, may include formal dual task testing and cognitive reserve questionnaires to assess the participant's cognitive reserve. If it is indicated that the participant's cognitive reserve is impaired, interventions that might promote and enhance cognitive reserve may be recommended.

### Corrective Action

As discussed herein, techniques for correcting environmental interference/distraction or performance deficits can be placed into two example categories: the first category is trying to make a recommendation to do something different before or during the assessment, and the second category is correcting the received sensor signal(s) after the assessment and/or during analysis.

For example, if a microphone detects high ambient noise, the solution, as described herein, may ask the participant to move to a different place before beginning the assessment. Similarly, if the camera detects low ambient light, the participant may be instructed, by the solution, to adjust the participant's lighting until it is satisfactory.

As another example, if the participant is not able to fix the issue, it may be possible to leverage traditional signal processing techniques to process out and remove aberrant signals. In particular, if a participant drops or throws the device, a high-amplitude, high-frequency signal may be generated across all target sensors, such as the accelerometer, the gyroscope, the microphone, and the video camera. This high-amplitude, high-frequency signal may be processed out and removed, by the solution, using a simple low-pass filter tuned for each sensor. Furthermore, the user can be queried, by the solution, to ask for confirmation that the participant dropped or threw the device.

In addition to previous issues, environmental conditions may be satisfactory at the start of an assessment, but may deteriorate throughout the assessment. In such a case, the solution may pause the assessment so that the participant can correct the newly introduced issue. For example, if there is suddenly an increase in ambient noise due to traffic outside the participant's window, the solution may pause the assessment until the traffic clears and/or until the participant closes the window.

There may be cases where, despite corrective actions before and during the assessment, artifacts may still be introduced by the environment. In such cases, to ameliorate the signal, the approach may depend on the modality of the artifact. For example, common mode rejection is an established technique to reduce ambient noise in a recording, but it may not be relevant for improving lighting conditions in a video recording.

The solution, as described herein, may adjust the scoring if there is a valid way to do so, or the solution may mark an assessment result as low quality, prompting an interpreter of the results to review the assessment carefully and possibly manually score the assessment.

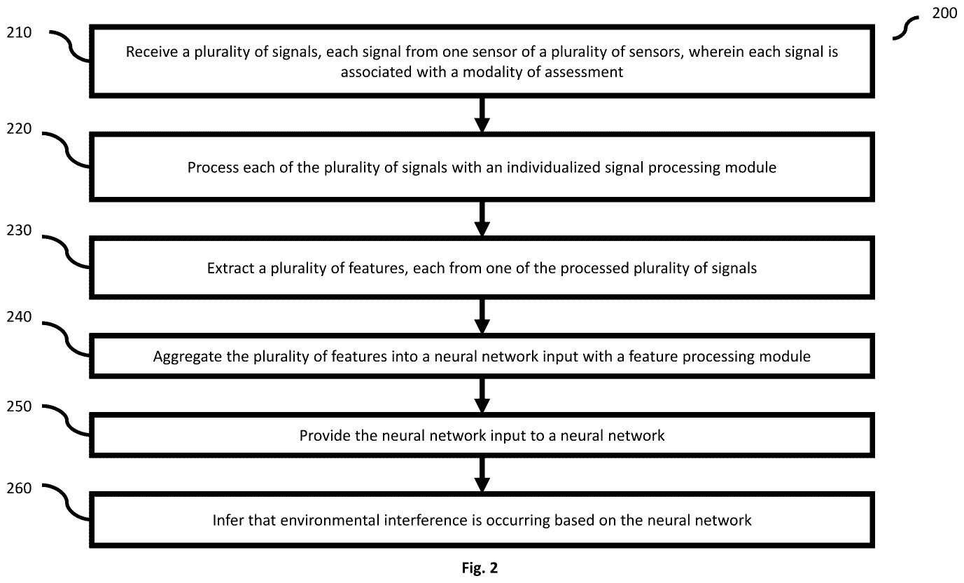

FIG. 2 is a flow diagram of example technique 200 for assessing an individual. At 210, a plurality of signals, each signal from one sensor of a plurality of sensors, are received. Each signal is associated with a modality of assessment. The plurality of sensors may include at least two of an accelerometer, a gyroscope, a microphone, and a video camera. At 220, each of the plurality of signals is processed with an individualized signal processing module. The processing of each of the plurality of signals may include transforming each of the plurality of signals. Transformation of each of the plurality of signals may include performing a Discrete Fourier Transform (DFT) on each of the plurality of signals. At 230, a plurality of features, each from one of the processed plurality of signals, are extracted. At 240, the plurality of features are aggregated into a neural network input with a feature processing module. The feature processing module may include a convolutional neural network. The neural network may include a recurrent neural network. In various embodiments, the plurality of features are aggregated into a neural network input for each time instance in a window of time instances. At 250, the neural network input is provided to a neural network. The neural network input may be provided to the neural network for each time instance in a window of time instances. At 260, that environmental interference is occurring is inferred based on the neural network. In some embodiments, a corrective action may be suggested based on the environmental interference. The techniques described in FIG. 2 may operate on one or more aspects of a computing node described herein. Although various neural network input(s) and/or various neural network(s) are referenced herein, it should be understood that, in place of these, any machine learning input and machine learning model/algorithm, respectively, may be used without departing from the scope and spirit of what is described herein.

The diagnostic solution, as presented herein, considers multiple modalities, increases accuracy of diagnostics, and allows for improved inference of device context. The diagnostic solution may be capable of correlating potential distractions across multiple sensing modalities using a single, overarching algorithm, which can simultaneously process multiple incoming sensor signals. The solution as described herein can operate to measure contextual awareness from low-level sensor data and detect human physical activities. The solution as described herein may operate on multiple modalities for a variety of problems, such as gait detection, fine-grain physiologic monitoring, and/or the like.

The solution, as described herein, may have advantages over conventional systems and solutions. In particular, unlike conventional solutions, the solution described herein is able to more fully assess contextual awareness during remote assessments with multimodal signals. The solution is also able to perform assessments for problems that make use of multiple modalities. In addition, the solution described herein may be more robust, more efficient, and more likely to provide accurate assessments compared to conventional solutions. Additionally, as compared to conventional solutions and systems, the solution described herein is capable of seamless integration and use with existing assessment systems, solutions, and tools. The solution described herein may make existing systems that perform assessments more robust, more efficient, and more likely to provide accurate results. Moreover, the solution described herein is capable of handling data and datasets that are typically very large and complex. The solution described herein is also able to gather and efficiently process data that is typically difficult to gather and store using any conventional techniques.

Although neural network machine learning models/algorithms, deep neural network machine learning models/algorithms and recurrent neural network machine learning models/algorithms are referenced herein, it should be understood that any machine learning model/algorithm may be used without departing from the scope and spirit of what is described herein. In particular, in various embodiments, the machine learning models may include an artificial neural network. In various embodiments, the machine learning models/algorithms, such as artificial neural networks described herein, may comprise a feedforward neural network, a radial basis function network, a self-organizing map, learning vector quantization, a recurrent neural network, a Hopfield network, a Boltzmann machine, an echo state network, long short term memory, a bi-directional recurrent neural network, a hierarchical recurrent neural network, a stochastic neural network, a modular neural network, an associative neural network, a deep neural network, a deep belief network, a convolutional neural networks, a convolutional deep belief network, a large memory storage and retrieval neural network, a deep Boltzmann machine, a deep stacking network, a tensor deep stacking network, a spike and slab restricted Boltzmann machine, a compound hierarchical-deep model, a deep coding network, a multilayer kernel machine, a deep Q-network, and/or the like. The machine learning models/algorithms described herein may additionally or alternatively comprise weak learning models, linear discriminant algorithms, logistic regression, and the like. The machine learning models/algorithms described herein may include supervised learning algorithms, unsupervised learning algorithms, reinforcement learning algorithms, and/or a hybrid of these algorithms.

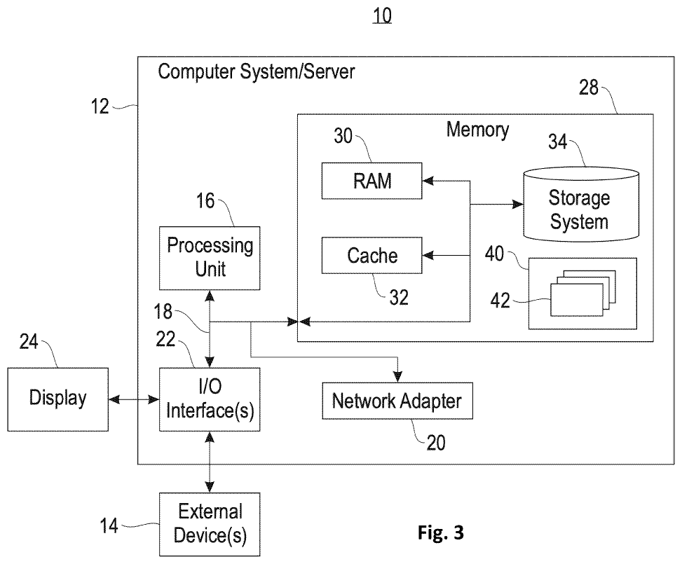

As shown in FIG. 3, computer system/server 12 in computing node 10 is shown in the form of a general-purpose computing device. For example, one or more computing nodes 10, with all or some of the components shown in FIG. 3 and described herein may be used as part of a cloud computing system. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, Peripheral Component Interconnect Express (PCIe), and Advanced Microcontroller Bus Architecture (AMBA).

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a drive, such as for reading from and writing to a removable, non-volatile drive, such as a USB drive, and/or a hard drive, such as an optical disk drive, for reading from or writing to a non-volatile optical drive or other media, such as optical media, can be provided. In such instances, each can be connected to bus 18 by one or more data media interfaces. As will be further depicted and described below, memory 28 may include at least one program product having a set (e.g., at least one) of program modules that are configured to carry out the functions of embodiments of the disclosure.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

The present disclosure may be embodied as a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a memory stick, a mechanically encoded device such as punch-cards or raised structures in a groove having instructions recorded thereon, and any suitable combination of the foregoing. A computer readable storage medium, as used herein, may be signals, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method for assessing environmental context around an individual taking an assessment, the method comprising:
receiving a plurality of signals, each signal from one sensor of a plurality of sensors, wherein each signal is associated with a modality of assessment;
processing each of the plurality of signals with an individualized signal processing module;
extracting a plurality of features, each from one of the processed plurality of signals;
aggregating the plurality of features into a machine learning input with a feature processing module;
providing the machine learning input to a machine learning algorithm;
inferring that environmental interference is occurring based on the output of the machine learning algorithm.

2. The method of claim 1, wherein the plurality of sensors includes at least two of an accelerometer, a gyroscope, a microphone, and a video camera.

3. The method of claim 1, wherein the processing of each of the plurality of signals comprises transforming each of the plurality of signals.

4. The method of claim 3, wherein the transforming each of the plurality of signals comprises performing a Discrete Fourier Transform (DFT) on each of the plurality of signals.

5. The method of claim 1, wherein the feature processing module comprises a convolutional neural network.

6. The method of claim 1, wherein the machine learning algorithm comprises a recurrent neural network.

7. The method of claim 1, wherein the machine learning input is provided to the machine learning algorithm for each time instance in a window of time instances.

8. The method of claim 7, wherein the aggregating the plurality of features into a machine learning input occurs for each time instance in a window of time instances.

9. The method of claim 1, further comprising suggesting a corrective action for the environmental interference by flagging or correcting a received signal.

10. The method of claim 1, further comprising suggesting a corrective action to negate an effect of the environmental interference by providing a recommendation to modify the environmental context.

11. The method of claim 1, further comprising:
determining a number of times that a device is dropped, wherein the device includes at least one sensor of the plurality of sensors;
recording movement of the individual from at least one sensor of the plurality of sensors; and
determining a manual dexterity of the individual based on the number of times that the device is dropped and the recorded movement.

12. The method of claim 1, further comprising calculating, based on the output of the machine learning algorithm, a total score of the environmental context, an effect of the environmental context on the individual, and a score of the assessment.

13. The method of claim 1, wherein the output comprises a quantitative environmental score and a qualitative environmental score.

14. The method of claim 13, wherein the qualitative environmental score indicates one or more of a degree of distraction for a particular interference, a potential degree of impact on the individual to perform the assessment, and an ability to process the plurality of signals compared to processing under an optimal set of conditions.

15. A system comprising:
a computing node comprising a computer readable storage medium having program instructions embodied therewith, the program instructions executable by a processor of the computing node to cause the processor to perform a method comprising:
receiving a plurality of signals, each signal from one sensor of a plurality of sensors, wherein each signal is associated with a modality of assessment;
processing each of the plurality of signals with an individualized signal processing module;
extracting a plurality of features, each from one of the processed plurality of signals;
aggregating the plurality of features into a machine learning input with a feature processing module;
providing the machine learning input to a machine learning algorithm;
inferring that environmental interference is occurring based on the output of the machine learning algorithm.

16. The system of claim 15, wherein the plurality of sensors includes at least two of an accelerometer, a gyroscope, a microphone, and a video camera.

17. The system of claim 15, wherein the processing of each of the plurality of signals comprises transforming each of the plurality of signals.

18. The system of claim 17, wherein the transforming each of the plurality of signals comprises performing a Discrete Fourier Transform (DFT) on each of the plurality of signals.

19. The system of claim 15, wherein the feature processing module comprises a convolutional neural network.

20. A computer program product for assessing environmental context around an individual taking an assessment comprising a computer readable storage medium having program instructions embodied therewith, the program instructions executable by a processor to cause the processor to perform a method comprising:
receiving a plurality of signals, each signal from one sensor of a plurality of sensors, wherein each signal is associated with a modality of assessment;
processing each of the plurality of signals with an individualized signal processing module;
extracting a plurality of features, each from one of the processed plurality of signals;
aggregating the plurality of features into a machine learning input with a feature processing module;
providing the machine learning input to a machine learning algorithm;
inferring that environmental interference is occurring based on the output of the machine learning algorithm.
